(19) United States
(12) Patent Application Publication
(10) Pub. No.: US 2010/0310633 A1
(43) Pub. Date: Dec. 9, 2010

(54) CHITINOSANASE

(75) Inventors: Bruno Moerschbacher, Muenster (DE); Nour Eddine El Gueddari, Muenster (DE); Markus Kohlhoff, Belo Horizonte (BR)

Correspondence Address: PATENT LAW OFFICES OF DR. NORMAN B. THOT, POSTFACH 10 17 56, RATINGEN, 40837, DE

(73) Assignee: Westfaelische Wilhelms-Universitaet Muenster, Muenster (DE)

(21) Appl. No.: 12/867,059

(22) PCT Filed: Feb. 16, 2009
(86) PCT No.: PCT/EP09/51822

§ 371 Date: Aug. 11, 2010

(30) Foreign Application Priority Data
Feb. 15, 2008 (EP) 08101693.3

(51) Int. Cl. A61L 15/00; C12N 9/14; C07H 21/04; C12N 15/63; C12N 5/10; A61K 38/46; C12P 19/04; C07H 1/00

(52) U.S. Cl. 424/445; 435/195; 536/23.2; 435/320.1; 435/325; 424/94.6; 435/101; 536/123.1

(57) ABSTRACT

A chitinosanase obtainable from the fungus Alternaria alternate. The chitinosanase specifically cleaves a GlcNAc-GlcN glycosidic bond in a chitosan, possesses a relative molecular weight as determined by SDS-PAGE of about 18 kDa, has an optimum pH of about 4 and has an optimum temperature of about 70° C.

### CROSS REFERENCE TO PRIOR APPLICATIONS

This application is a U.S. National Phase application under 35 U.S.C. §371 of International Application No. PCT/EP2009/051822, filed on Feb. 16, 2009 and which claims benefit to European Patent Application No. 08101693.3, filed on Feb. 15, 2008. The International Application was published in German on Aug. 20, 2009 as WO 2009/101213 A1 under PCT Article 21(2).

### FIELD

The present invention relates to a chitosan-degrading enzyme from the fungus Alternaria alternata, designated as chitinosanase, which specifically cleaves the GlcNAc-GlcN glycosidic bond in chitosan, DNA sequences that encode this enzyme, vectors and host cells with this DNA sequence, the production of this enzyme, and use thereof for the cleavage of chitosan.

### SEQUENCE LISTING

The Sequence Listing associated with this application (SEQ ID NOs:1 and 2 Chitinosanase fragments) is filed in electronic form via EFS-Web and hereby incorporated by reference into this specification in its entirety. The name of the text file containing the Sequence Listing is Sequence_Listing. The size of the text file is 754 Bytes, and the text file was created on Aug. 10, 2010.

### BACKGROUND

Chitosan is a linear copolymer from glucosamine (GlcN, D) and N-acetyl-glucosamine (GlcNAc, A). Chitosan is produced commercially from chitin, a fully acetylated GlcNAc polymer produced either by partial de-N-acetylation or by complete de-N-acetylation and subsequent partial re-N-acetylation. In both eases, owing to the chemical procedure, partially acetylated chitosans are obtained with a random distribution of the acetyl residues along the linear main chain.

| Chitin | DA |
|---|---|
| AAAAAAAAAAAAAAAAAAAAAAAAAAAAAAAAAAAAA | 100% |
| PolyGlucosamine | DA |
| DDDDDDDDDDDDDDDDDDDDDDDDDDDDDDDDDD | 0% |
| Chitosan (for example) | DA |
| DDDAADDDADAADAAADDAADDAAADDADDDADAAA | 50% |

The polymer can be depolymerized physically, chemically or enzymatically. Physical depolymerization is effected by ultrasonic treatment, and its specific character, with respect to the point at which the polymer is fragmented, is still unknown. Chemical depolymerization normally takes place purely randomly (random acid hydrolysis) or, if working under precisely defined conditions, behind each “A” (partial acid hydrolysis) or behind each “D” (oxidative deamination), the D unit being deaminated to anhydro-mannose (M). For the above chitosan we would have:

random acid hydrolysis DDDA ADD DAD AADA AAD DA ADDA AA DDA D DDA DA AA

partial acid hydrolysis DDDA A DDDA DA A DA A A DDA A DDA A A DDA DDDA DA A A

oxidative deamination M M M AAM M M AM AAM AAAM M AAM M AAAM M AM M M AM AAA

Enzymatic depolymerization can either be effected with chitinases (hereinafter “chitin.”) or with chitosanases (hereinafter “chitos.”), All chitinases cleave between “AA”, some additionally also between “AD” or “DA”. All chitosanases cleave between “DD”, some additionally also between “DA” or “AD”. For the above chitosan we would have:

Chitin. AA DDDA ADDDADA ADA A ADDA ADDA A ADDADDDADA AA

Chitin. AA/AD DDDA A DDDA DA A DA A A DDA A DDA A A DDA DDDA DA A A

Chitin. AA/DA DDD A ADDD AD A AD A A ADD A ADD A A ADD ADDD AD A A A

Chitos. DD D D DAAD D DADAADAAAD DAAD DAAAD DAD D DADAAA

Chitos. DD/DA D D D AAD D D AD AAD AAAD D AAD D AAAD D AD D D AD AAA

Chitos. DD/AD D D DAA D D DA DAA DAAA D DAA D DAAA D DA D D DA DAAA

Provision of a chitosanase with this defined substrate specificity would be highly desirable.

A chitosan-degrading enzyme was isolated from the fungus Alternaria alternata and characterized, and it was found that this enzyme has a substrate specificity not previously described, which clearly differentiates it from the previously known chitinases and chitosanases. The enzyme found has an absolute specificity for the cleavage of “AD”. It is accordingly neither a typical chitinase, which have in common the ability to cleave “AA”, nor a typical chitosanase, as these can all cleave “DD”. The enzyme thus defines a new class of chitosan-hydrolyzing enzymes, which we designate here as “chitinosanases” (chitinos.). The degradation products of the above chitosan would be:

Chitinos. AD DDDAA DDDA DAA DAAA DDAA DDAAA DDA DDDA DAAA

Even this very limited example shows: the various methods produce very different product mixtures. There is in fact even greater variety, as the specificities of the enzymes are shown simplified. Thus, it was not taken into account that the sugar unit can also be decisive, as well as the actual cleavage site (for example, human lysozyme cleaves “AAA” to “AA A”), that often the specificities are not absolute but only partial, and that many enzymes require a minimum oligomer length to accept this as substrate (thus, most of the chitinases cleave “AAAA” into two molecules “AA” or into one of each molecule “A” and “AAA”, but the products of this degradation, i.e. the trimer “AAA” and the dimer “AA”, cannot be cleaved further). What is striking is that all product mixtures, regardless of whether they are produced chemically or enzymatically with chitinases or chitosanases, are characterized by high complexity of the product mixture.

To illustrate this, and to make it easier to compare the products from the different methods, the oligomeric products should be sorted according to size:

Random acid hydrolysis D DA DA AA AA DAD ADD DDA DDA AAD DDDA ADDA AADA

Partial acid hydrolysis A A A A A A A A A DA DA DA DDA DDA DDA DDDA DDDA DDDA

Oxidative deamination M M M M M M M M M M AM AM AM AAM AAM AAM AAA AAAM AAAM

Chitin, AA A A A A ADA DDDA ADDA ADDA ADDDADA ADDADDDADA

Chitin. AA/AD A A A A A A A A A DA DA DA DDA DDA DDA DDDA DDDA DDDA

Chitin. AA/DA A A A A A A A A A A AD AD AD DDD ADD ADD ADD ADDD ADDD

Chitos. DD D D D D DAD DAAD DAAD DAAAD DADAAA DADAADAAAD

Chitos. DD/DA D D D D D D D D D D AD AD AD AAD AAD AAD AAA AAAD AAAD

Chitos. DD/AD D D D D D D D D D DA DA DA DAA DAA DAA DAAA DAAA DAAA

Chitinos. AD DDA DAA DDDAA DDDA DDDA DDAA DAAA DAAA DDAAA

It is evident that in nearly all methods, very small products result, and these are often also predominant. Chitinosanase is an exception. Furthermore, larger oligomers are only produced when using highly specific enzymes, which only cleave one type of bond, whether it is “AA”, “DD” or “AD”. It seems, however, that for specific interactions with proteins (for example, enzymes, receptors, etc.) and therefore for biological activities, a minimum size of four, or better still, five sugar residues is necessary, for larger oligomers to be of biotechnological and biomedical interest.

On closer examination of the resultant oligomers, it can be seen that the products of partial acid hydrolysis as well as the products of chitinase AA/AD can be described with the formula DₙA, and those of chitinase AA/DA with the formula ADₙ, so in these three cases we are dealing with glucosamine oligomers with a single acetyl group, located either at the reducing end or on the nonreducing end. The products of oxidative deamination correspond to the formula AₙM, those of chitosanases DD/DA and DD/AD correspond to the formulas AₙD, and DAₙ, and so in these three cases we have N-acetyl-glucosamine oligomers that lack an acetyl group either at the reducing end or on the nonreducing end. The products of random acid hydrolysis do not exhibit any kind of regularity, those of chitinase AA and of chitosanase DD only exhibit the presence of A or D residues on both ends of the chain, though in both cases the presence and the distribution of A and D residues in the middle of the oligomers is almost random (except that in the products of chitinase AA there are only individual A residues and in those of chitosanase DD there are only individual D residues).

In contrast, chitinosanase gives partially acetylated oligomers of the formula DₙAₘ, in which all D residues occur as a block on the nonreducing end, and all A residues as a block on the reducing end. Such oligomers cannot be produced with any previously known method, with the possible exception of very expensive chemical synthesis.

### SUMMARY

In an embodiment, the present invention provides for a chitinosanase obtainable from the fungus Alternaria alternate. The chitinosanase specifically cleaves a GlcNAc-GlcN glycosidic bond in a chitosan, possesses a relative molecular weight as determined by SDS-PAGE of about 18 kDa, has an optimum pH of about 4 and has an optimum temperature of about 70° C.

### BRIEF DESCRIPTION OF THE FIGURES

The present invention is described in greater detail below on the basis of embodiments and of the drawings in which:

FIG. 1 shows the purification of chitinosanase;

FIG. 1a shows a FPLC chromatogram (column: Mono S; sample: concentrated medium of A. alternate (0.1% w/v); buffer: 50 mM Na-acetate pH 4; flow rate 0.25 ml/min; elution: 0-1 M NaCl; fractions: 1 ml);

FIG. 1b shows adot-assay of the FPLC fractions (substrate: chitosan DA 64%);

FIG. 1c shows a SDS-PAGE (12%) of fraction 10 with (A) Coomassie staining and (B) zymogram (substrate: chitosan DA 64%);

FIG. 2 shows an optimum pH (A), optimum temperature (B) and temperature stability (C) of chitinosanase. A: buffer used (in each case 50 mM): pH 1.5-2.5: glycine/Cl, pH 3.0-7.0: citrate/phosphate, pH 8.0-9.0. Tris/Cl, pH 9.0-11.0: carbonate; incubation: 2 h at 60° C.; B: incubation: 2 h in 50 mM Na-acetate pH 4.3; C: storage at 4° C. (squares), 37° C. (circles), 60° C. (triangles) or 80° C. (trapeziums); incubation: 2 h at 60° C. in 50 mM Na-acetate pH 4.3. Chitosan DA 66% was used as substrate in all the experiments;

FIG. 3 shows a substrate specificity of chitinosanase. Enzyme: 4.5 pkat purified chitinosanase; substrate: 20 μg chitosan of the respective DA or glycol-chitin (for DA 100%); incubation: 15 h at 37° C. in 50 mM Na-acetate pH 4.3;

FIG. 4 shows a mass spectra of the products of chitinosanase degradation of chitosans with different DA (%). Incubation: 4.5 pkat purified chitinosanase; 15 h at 37° C. in 20 mM Na-acetate pH 4.3; analysis: MALDI-TOF-MS; products are designated DxAy; x=number of GlcN, y: number of GlcNAc;

FIG. 5 shows an NMR analysis of the chitinosanase products. Incubation: 50 pkat purified chitinosanase, 24 h at 37° C. in 50 mM citrate pH 4.3; analysis: 400 MHz ¹H-NMR, For comparison, a partially acid-hydrolyzed chitosan was analyzed (top);

FIG. 6 shows a TLC analysis of the chitinosanase products. Substrate: fully deacetylated tetramer, GlcN₄=D₄ (top), fully acetylated hexamer, GlcNAc₆=A₆ (bottom); incubation: 50 pkat purified chitinosanase, 0-240 min or overnight (O.N.) at 37° C. in 50 mM citrate pH 4.5; analysis: solvent 28% ammonia/propan-1-ol (1:2, v/v); staining: ninhydrin (GlcN) or aniline-diphenylamine (GlcNAc); as control, the substrate was incubated overnight without enzyme (c); the respective oligomers and monomers served as standards (s); and

FIG. 7 shows an actual (experiment) and expected relative frequency of the fully acetylated dimer (A₂) as product of chitinosanase degradation of a chitosan polymer with DA 66% on the assumption of different strengths of side activities for GlcNAc→GlcNAc bond cleavage.

### DETAILED DESCRIPTION

The present invention therefore relates to

a chitinosanase, obtainable from the fungus Alternaria alternata, and which:

(a) specifically cleaves the GlcNAc-GlcN (A-D) glycosidic bond in chitosan,

(b) has a relative molecular weight, determined by SDS-PAGE, of about 18 kDa,

(c) has an optimum pH of about pH 4 and

(d) has an optimum temperature of about 70° C.;

(2) a chitinosanase, in particular an embodiment of chitinosanase (1), which

with an amino acid sequence, which has one or both of the protein fragments shown in SEQ ID NO: 1 and 2,

is a sequence homolog of (a), which has similarity of at least 80% to sequence (a),

is a fragment of (a) or (b), which has at least 10 consecutive amino acid residues of sequence (a) or (b), or

is a derivative of (a), (b) or (c);

a DNA sequence that encodes a chitinosanase according to (1) or (2);

a vector that contains a DNA sequence according to (3);

a host cell that has been transformed/transfected with the vector according to (5) and/or has the DNA sequence according to (3);

a method of production and a chitinosanase according to (1) or (2), comprising the cultivating of a host cell according to (5) and isolating the chitinosanase from the cultivated host cells and/or from the culture solution;

an enzyme composition or pharmaceutical composition containing a chitinosanase according to (1) or (2);

a method of degradation of chitosan, comprising reacting the chitosan with a chitinosanase according to (1) or (2), with a host cell according to (5) or with an enzyme composition according to (7) and isolating the chitosan degradation products;

chitosan degradation products obtainable by a method according to (8);

the use of a chitinosanase according to (1) or (2) or of an enzyme composition according to (7) for the production of a wound dressing; and

a method of wound treatment comprising applying a wound dressing, which contains a chitinosanase according to (1) or (2) and a (partially acetylated) chitosan, on a patient's wound.

In an embodiment of the present invention, the chitinosanase has the following properties:

The chitinosanase specifically cleaves the GlcNAc-GlcN (A-D) glycosidic bond in chitosan. “Specifically” means, in the sense of the present invention, that there is no side activity with respect to DD cleavage or only a slight side activity (for example, <4%) with respect to AA cleavage (see FIGS. 3 and 7). The specificity of the chitosanase according to the present invention for the cleavage of A-D is therefore greater than 95%, for example greater than 98%.

The chitinosanase has a relative molecular weight, as determined by SDS-PAGE, of about 18 kDa (for example, 18 kDa) and for the specific cleavage of A-D an optimum pH of about pH 4 (for example pH 4) and an optimum temperature of about 70° C. (for example 70° C.).

The chitinosanase according to an embodiment of the present invention can, for example, be an enzyme that is obtainable from the Alternaria alternata strain CCT 2816 of the Coleção de Culturas Tropical, Brazil (deposited according to the Budapest Treaty as DSM 22279).

In an embodiment of the present invention, the chitinosanase can, for example, possess an amino acid sequence that comprises one or both protein fragments of SEQ ID NO:1 and 2.

In an embodiment of the present invention, the sequence homologs of the chitinosanase can have a similarity of at least 80%, for example at least 90%, or of at least 95% or at least 98%. This includes conservative exchanges of individual or several consecutive amino acid residues, the deletion of individual or several consecutive amino acid residues and the addition/insertion of individual amino acid residues or several consecutive amino acid residues.

In an embodiment of the present invention, fragments of the chitinosanase or of sequence homologs can have at least 10 consecutive amino acid residues of the starting sequence.

In an embodiment of the present invention, derivatives of the chitinosanase or of the sequence homologs or of the fragments thereof comprise both condensation products with other functional protein or peptide structures (for example, other enzymes, antibodies, secretion proteins, sequences for purification of the enzyme etc., which can be joined directly or via a linker to the chitinosanase), and with low-molecular organic residues (for example, C- and N-terminal residues, protecting groups, markers etc.) with solid phases (for example, microtiter plates, beads etc.).

In an embodiment of the present invention, the DNA sequence comprises both DNA and RNA sequences. Variations of the sequences that have a homology of at least 80%, for example at least 90%, or at least 98% with the starting sequence, are also included.

In an embodiment of the present invention, the vector can, for example, be a transfection vector among other things, which in addition to the sequence can also contain other functional sequences such as promoters, selection marker sequences etc.

In an embodiment of the present invention, the host cell which has been transformed/transfected with the vector according to an embodiment of the present invention and/or has the DNA sequence according to an embodiment of the present invention is a eukaryotic cell (fungus, yeast, mammalian cell etc.) or a prokaryotic cell (E. coli etc.).

In an embodiment, the present invention also provides a method of producing a chitinosanase according to an embodiment hereof which method comprises cultivating a host cell as defined above and isolating the chitinosanase from the cultivated host cells and/or from the culture solution. The method can moreover comprise suitable purification steps and/or reactions of the chitinosanase obtained initially.

In an embodiment of the present invention, the enzyme composition can, depending on the field of application of the composition, contain not only further enzymes (such as glucosaminidase) but also excipients such as stabilizers, buffers etc.

In an embodiment of the method of the present invention, the degradation of chitosan comprises the direct reaction of the chitosan with a chitinosanase according to the first or second embodiment of the present invention or with an enzyme composition according to an embodiment of the present invention. Alternatively, a host cell according to an embodiment of the present invention can also be used, which produces the chitinosanase in situ. Other enzymes can be used in addition to the chitinosanase of the present invention. The method further comprises the purification of the resultant degradation products and the modifications as described hereunder.

The pharmaceutical composition according to the aforementioned embodiment of the present invention can be a component of a wound dressing. Said wound dressing can, for example, also contain a (partially acetylated) chitosan as well as the chitinosanase according to the present invention.

In an embodiment of the present invention, chitosan degradation products produced by the chitinosanase according to the present invention differ from the oligomers presented above. They are characterized in that can contain any number of A and D residues, however, all D residues are concentrated toward the nonreducing end, and all A residues are concentrated toward the reducing end. The formula of the products is accordingly DₙAₘ. Thus, they are partially acetylated chitosan oligomers with a block distribution of the acetyl residues, with a glucosamine oligomer block on the nonreducing end and an N-acetyl-glucosamine oligomer block at the reducing end. In contrast to other larger oligomers, the architecture of each oligomer is therefore described unambiguously by specifying n and m. These two values are determined by mass spectroscopy.

The products of chitinosanase degradation, in contrast to those from any other method, can be purified relatively easily. In a first step, the oligomers can be separated by size exclusion chromatography according to their degree of polymerization. By this method, fully deacetylated glucosamine oligomers Dₙ can be separated from fully acetylated N-acetyl-glucosamine oligomers Aₙ₋₁, which are smaller by one residue. The individual oligomer mixtures can then be separated in a second step by cation exchange chromatography according to their charge density, and accordingly according to the number of acetyl residues present. For example, in the first step, the tetramers DDDA, DDAA and DAAA are separated from the pentamers DDDDA, DDDAA, DDAAA and DAAAA, and in the second step, the three tetramers or the four pentamers, respectively, are separated from one another. This is the first method to permit the production of these partially acetylated chitosan oligomers with precisely known architecture.

Such oligomers might on the one hand have interesting and new bioactivities, as it is assumed that the biological activity depends not only on the degree of polymerization (DP) and the degree of acetylation (DA), but also on the distribution pattern of the acetyl residues (pattern of acetylation, PA). On the other hand, they could also be used to obtain, by polymerization, partially acetylated chitosan polymers with a known pattern of acetylation. These could, for example, be constructed so that they are degraded by certain endogenous enzymes into defined products, or alternatively cannot be degraded by said enzymes. It would thus be possible on the one hand to control the degradation rate and therefore the time they remain in the body or tissue, and on the other hand biologically active oligomeric products could be released as required.

The first human enzyme with chitosanolytic activity was recently described in the doctoral thesis of Christian Gorzelanny; Westphalian Wilhelm University Münster, Germany. The human chitotriosidase cleaves chitin and partially acetylated chitosans sequence-specifically between two acetylated residues (it is thus a chitinase of the AA type). This provides evidence that both the degradation rate and the quantity and quality of the resultant degradation products of a partially acetylated chitosan, which is used for example as a component of a wound dressing, depend on the PA of the chitosan. At the same time, the resultant degradation products possess pro-inflammatory activity and therefore can positively support wound healing.

Knowledge about the substrate specificity of human chitotriosidase offers prospects for targeted engineering of partially acetylated chitosans with known DP, DA and PA. Such a chitosan could be designed so that it has a predictable degradation rate in a patient's body and releases particular bioactive chitosan oligomers with known kinetics. However, methods for production of chitosans with defined, nonrandom PA are not yet known (the existing chemical methods of production of partially acetylated chitosans yield random PA). By means of the chitinosanase described here, chitosan oligomers can be produced with nonrandom (but block) PA. These could be polymerized selectively in a second step, so as to produce chitosan polymers with regular PA and therefore precisely-defined degradation products in the target tissue.

Two basic possible methods exist for this polymerization, either a chemical route or an enzymatic route. Chemical synthesis of partially acetylated chitosan oligomers is still in its infancy, but is in principle possible. However, the costs are already enormous for the synthesis of dimers, and will probably increase disproportionately with chain length. At the same time, owing to the large number of steps required, yields decline considerably. Enzymatic polymerization might provide an alternative. However, no enzyme is known that would produce such oligomers in nature. The synthesis always appears to proceed via the polymerization of N-acetyl-glucosamine to chitin oligomers or polymers, which are then partially de-N-acetylated and optionally depolymerized. Many chitinolytic enzymes possess glycosyltransferase side activity. They can thus not only cleave glycosidic bonds, but also transfer sugar residues to others. One possibility is to use the chitinosanase described here to achieve polymerization in the back reaction. If necessary, the rate of this back reaction could be improved by suitable protein engineering.

The following applications exist for the chitosan degradation products according to an embodiment of the present invention, for example, for the chitosan oligomers with defined PA, such as block PA in the products of chitinosanase:

wound healing without scars (important, for example, for patients with a keloid formation tendency, burns);

pro- or anti-growth factor activity (for example, with respect to angiogenesis: pro-angiogenic important in wound healing, anti-angiogenic important for combating tumors or wet macular degeneration);

pro/anti-inflammatory (both important, for example, in wound healing, in particular in the case of chronic wounds such as in the case of bedridden patients or diabetic patients); and

antitumor activity.

These applications are possible similarly for the chitinosanases according to the embodiments of the present invention or the pharmaceutical composition according to another embodiment of the present invention, namely when the degradation products are produced directly in situ from enzyme and chitosan, for example, the pharmaceutical composition contains both stated components. This applies, for example, to wound treatment.

For the designer chitosans outlined above, the following fields of application may be mentioned: designer chitosans will be appropriate when the specificity of the chitosanolytic enzymes in a target tissue is known (for example, chitotriosidase, acidic mammalian chitinase AMCase or lysozyme in humans, but also corresponding enzymes, for example, in farm animals or in crop plants). In that case, it is possible to produce a tailor-made chitosan with known retention time or turnover rate, with known kinetics, quantity and quality of known bioactive or inactive degradation products etc. Designer chitosans could also have specific physicochemical properties, which could be important, for example, for the formation or stability of nanoparticles, hydrogels, films, solutions or for the coating of surfaces such as electrodes and/or implants.

The production of an engineered designer chitosan with inbuilt digestibility in human tissues can, for example, take place as follows:

hydrolyze chitosan polymer with medium DA with chitinosanase;

separate products by GPC;

select dimer (DA) and tetramer (DAAA DDAA DDDA) fraction;

further purify tetramer fraction by CEC, select DAAA;

polymerize dimers and tetramers by reverse chitinosanase reaction:

DADADADADADADADADADADAAADADADADADADADADAAADADAD ADADADADADADADADAAADADADADADADADADAAADADADADADADADADAD ADADADADADA

Product after lysozyme degradation (in vitro or in vivo):

| | |
|---|---|
| DADADADADADADADADADADAA | DP 23 |
| ADADADADADADADADAA | DP 18 |
| ADADADADADADADADADADADAA | DP 24 |
| ADADADADADADADADAA | DP 18 |
| ADADADADADADADADADADADADADADA | DP 29 |

The mean DP of the lysozyme products can, for example, be adjusted by means of the mixture ratio of the two oligomers during polymerization, as each tetramer incorporates one cleavage site. The medium DA of all products is 50%, with regular PA.

If the DP also requires precise adjustment, the polymerization must be carried out in several steps:

polymerize dimer, select, for example, DP 6 (hexamer) by GPC: DADADA;

polymerize hexamer and the aforementioned tetramer in 1/1 ratio, select DP 10 (decamer) by GPC: DADADADAAA and DAAADADADA;

incubate decamer with chitinase B from Serratia marcescens (exo-chitinase, which splits off AA-dimers from the reducing end): DADADADA, AA and DAAADADADA;

select decamer by GPC: DAAADADADA;

polymerize decamer: . . . DAAADADADADAAADADADADAAADADADA . . .

This polymer is cleaved by lysozyme to decamers:

. . . DAA ADADADADAA ADADADADAA ADADADA . . .

The particular substrate specificity also leads to the production of special products, namely partially acetylated chitosan oligomers with a precisely defined, block distribution of the acetyl residues. Until now there has been no method for producing such oligomers, which potentially have extremely interesting biological activities or can also be used as starting substances for the synthesis of novel polymers with definable properties.

The present invention will be explained in more detail with the following examples. These do not, however, restrict the invention in any way.

The Alternaria alternata strain CCT 2816 of the Coleção de Culturas Tropical was deposited on Feb. 11, 2009 at the DSMZ, Deutsche Gesellschaft für Mikroorganismen and Zellkulturen, Inhoffenstr. 7B, D-38124 Braunschweig, under the designation DSM 22279 according to the Budapest Treaty.

### EXAMPLES

### Example 1

### Purification and Characterization of the Chitinosanase

Cultivation of the fungus: Alternaria alternata (strain CCT 2816, Coleção de Culturas Tropical, Brazil) was cultivated as a permanent culture on sterile MA medium solidified with agar (malt extract 2%, agar 2%). Cultivation was first carried out for a week at 28° C. and with varying light conditions (12 h light, 12 h dark), which led to sporulation of the fungus. Then the plates were stored in the refrigerator. Refreshing was effected every 3 to 4 months.

Long-term storage was provided with an MA-agar filled slant tube, which was inoculated with mycelium and after a short growth period was covered with a layer of sterile paraffin oil.

To obtain a preliminary culture in liquid medium, 2% peptone and 2% glucose were added to MA medium without agar. Then two or three pieces, with size of 0.5 cm², of the Alternaria permanent culture were put in 50 ml of the liquid MA medium and incubated for five days at 28° C. in darkness. Eight flasks (each 500 ml) were inoculated with this preliminary culture and incubated once again for 5 to 7 d at 28° C. in darkness.

Enrichment: The culture medium was harvested by centrifugation at 13000 rpm. The supernatant was carefully removed and any residual mycelium was removed by vacuum filtration on a membrane filter (pore size 0.45 μm). The volume of the medium was determined and it was used for ultrafiltration. Ultrafiltration was carried out in an ultrafiltration cell (cubic capacity 500 ml) with a pressure of 3 bar. The membrane used had an exclusion molecular weight of 10 kDa. The culture medium of Alternaria alternate was thus concentrated from approx. 4 I to 100 ml. Then the 100 ml was freeze-dried in a pear-shaped distilling flask and the dry residue was taken up in 10 ml distilled water and used for gel filtration. This was carried out using PD 10 columns. The PD-10 columns were first equilibrated with 4×4 ml Na-acetate buffer (50 mM, pH 4.0) and in each case 2.5 ml of the culture medium concentrate was applied to the column and was eluted after percolation with 3.5 ml Na-acetate buffer. The enzymatic measurements and protein determination were then carried out with this eluate.

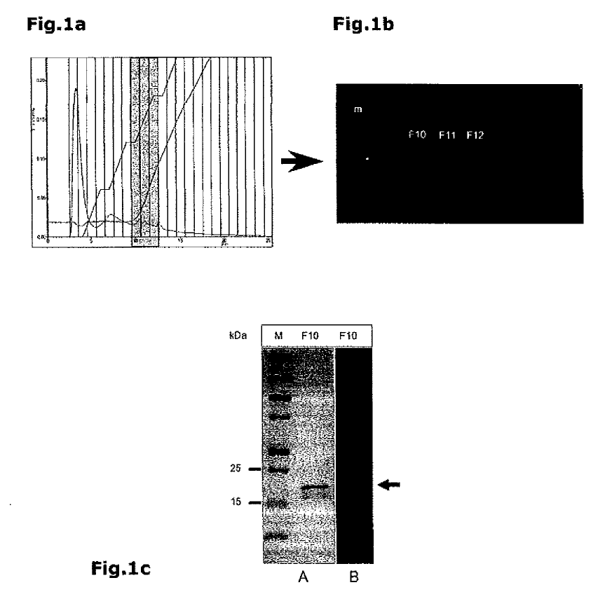

Purification: The chitinosanase was purified by FPLC based on the principle of cation exchange chromatography. The fractions were tested for chitosanolytic activity by a dot assay, active fractions were pooled and tested electrophoretically for purity (FIG. 1).

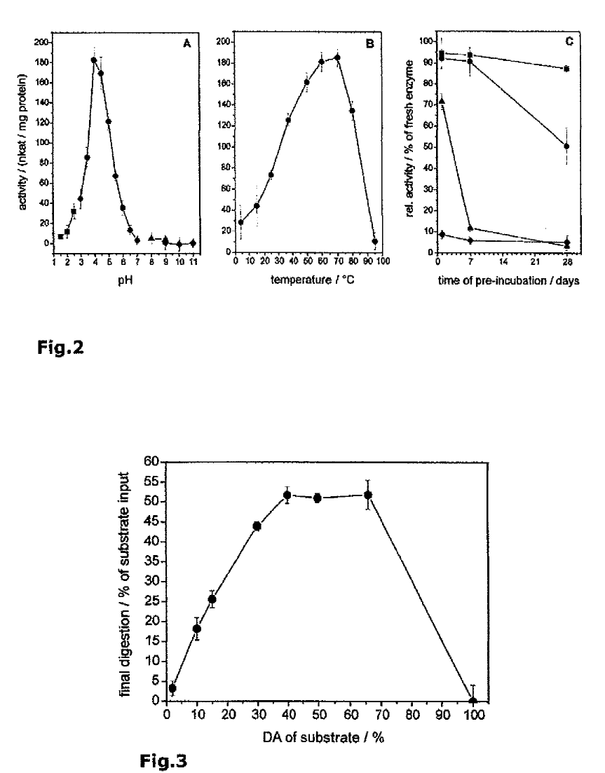

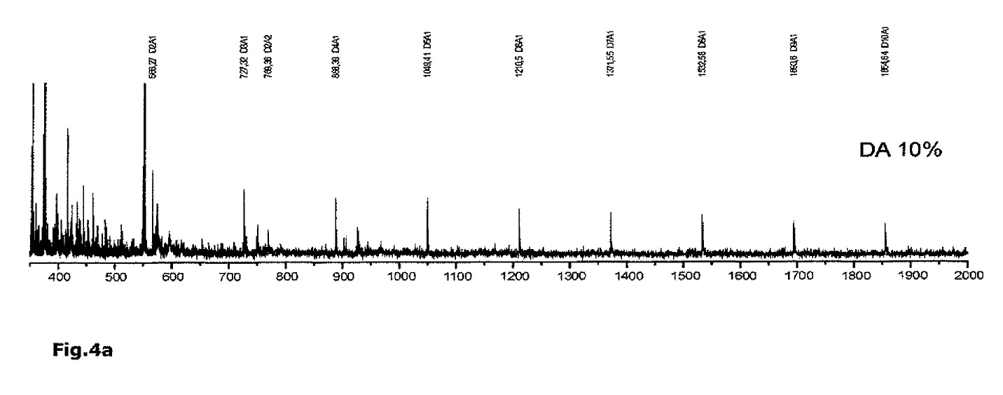

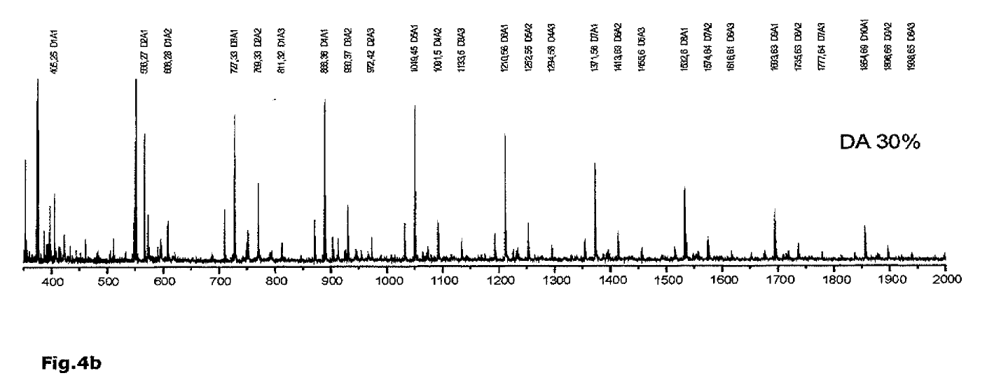

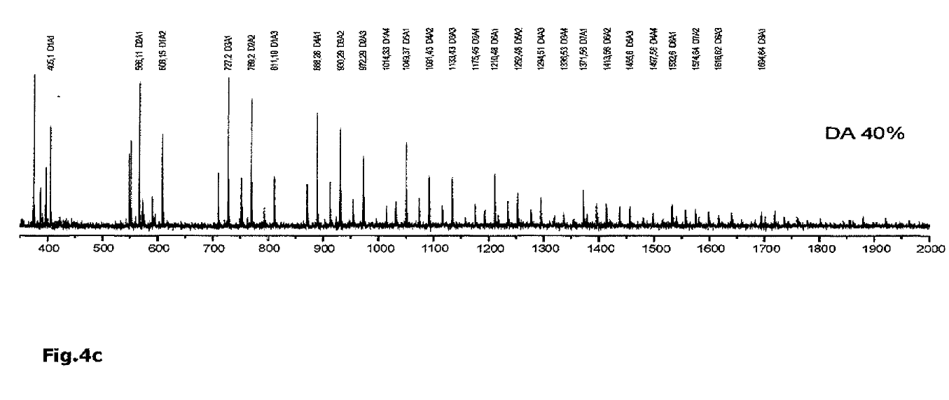

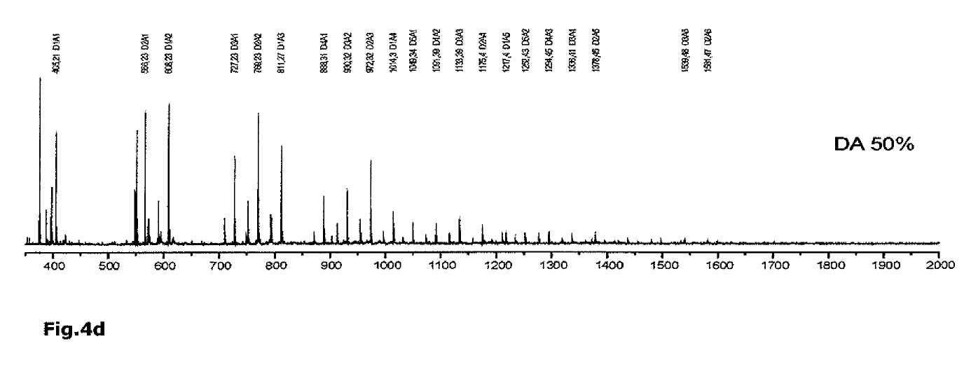

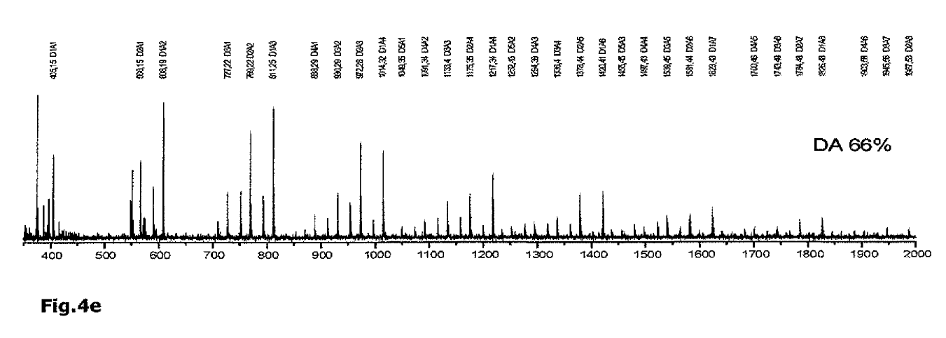

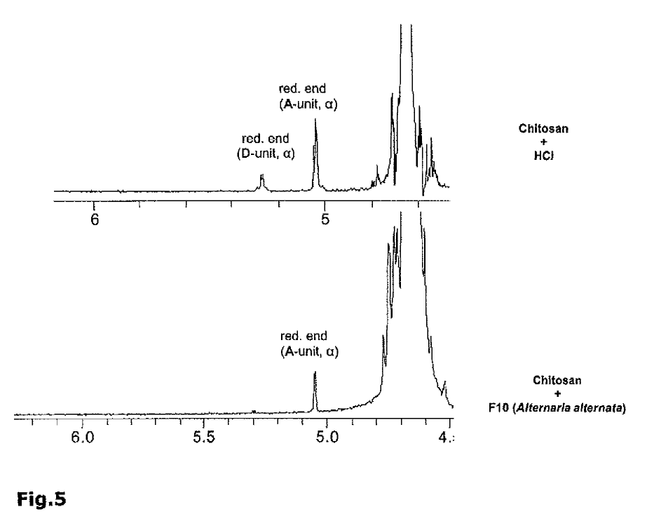

Properties of the chitinosanase: The purified enzyme was characterized by determining the relative molecular weight by SDS-PAGE (FIG. 1C), the optimum pH, the optimum temperature, the temperature stability (FIG. 2) and the substrate specificity for chitosans with different degrees of acetylation (FIG. 3). The relative molecular weight was 18 kDa. The optimum pH was pH 4 and the optimum temperature was 70° C. After one week at 37° C., 90% of the enzyme activity was still present, and even after storage for four weeks at this temperature, half the activity could still be detected. Chitosans with the medium degree of acetylation (DA) proved to be the most suitable substrates. The products of chitinosanase degradation of various substrates were analyzed by mass spectrometry (FIG. 4) and NMR (FIG. 5). Chitosan with low DA gave chitosan oligomers with only a single acetyl residue. With increasing DA of the substrate, products with several acetyl residues also increasingly appeared. All products bore an acetylated unit at the reducing end.

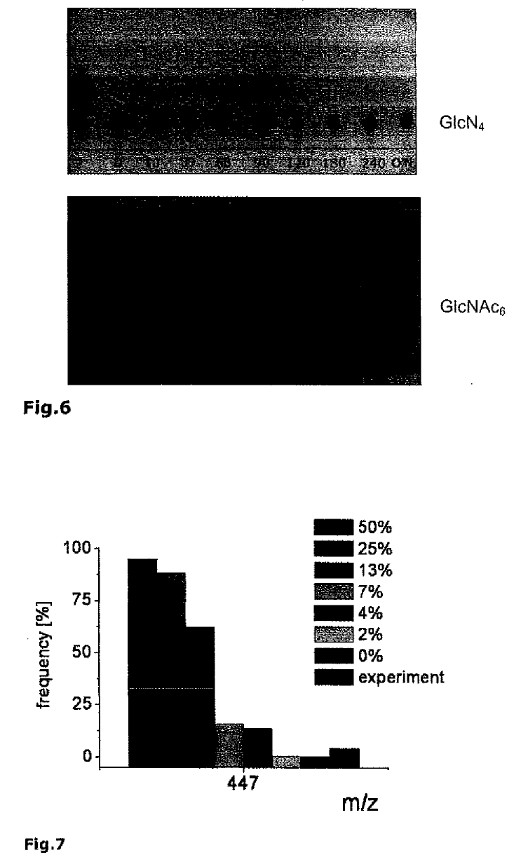

Fully acetylated chitin oligomers as well as fully deacetylated glucosamine oligomers were not degraded by the chitinosanase even with extensive incubation (FIG. 6). Therefore the enzyme cannot hydrolyze glycosidic bonds between two acetylated or between two deacetylated residues. As all the products bear an acetylated residue at the reducing end, it was concluded that chitinosanase can cleave the GlcNAc-GlcN glycosidic bond, but not the GlcN-GlcNAc bond.

Analysis of the products by mass spectrometry provides that fully acetylated or fully deacetylated products never occur. Degradation of a chitosan with low DA results exclusively in products with a single acetyl residue. With increasing DA of the substrate, even higher acetylated oligomers form, with only a single deacetylated residue. The fully deacetylated or fully acetylated regions of the respective products are apparently not degraded further.

The specificity of cleavage is also demonstrated by comparing the experimental MS spectra (FIG. 4) with the virtual MS spectra of a computer program (“Chitosan-Hydrolysator”), which can perform a sequence-specific hydrolysis of a virtual chitosan with any DP and DA (and random distribution PA of the acetyl residues) and can construct a theoretical MS spectrum of the products. If we assume a 100% specificity for the cleavage of the GlcNAc→GlcN bond, the virtual spectra obtained are almost identical to those obtained experimentally (MS analysis only conditionally provides quantitatively meaningful data, as different oligomers have different response factors). The lowest side activity for cleavage of the GlcN→GlcN bond would lead to degradation of the higher-molecular DₙA₁ products of the degradation of a chitosan with DA 10%; such side activity can thus certainly also be ruled out for polymeric substrates. Side activity for the GlcNAc→GlcNAc bond would more likely be noticeable in the degradation of a highly acetylated chitosan, it would lead to the production of the fully acetylated dimer (A₂), which with absolute specificity for the GlcNAc→GlcN bond is not to be expected in a measurable amount. With a side activity of 4%, the A₂ peak should already emerge clearly, whereas at a side activity of 2% it would still not be visible (FIG. 7). In fact, in the experimentally determined mass spectrogram, there is a very small peak at the relative mass m/z of 447 (=A₂). This is the only indicator for the presence of such side activity; it must be borne in mind, however, that the quantification of the dimer based on the matrix used in the MALDI-MS method is unreliable, and this might equally well be an artifact of the matrix. It can be asserted, however, that A₂ occurs as a product, if at all, then with lower frequency than would be expected at a side activity of 4%. Thus, even in the cleavage of partially acetylated chitosan polymers, chitinosanase has a high degree of specificity for cleavage of the GlcNAc→GlcN bond, and possibly this specificity is also really absolute.

### Example 2

### Sequencing

In order to digest the chitinosanase from Alternaria alternata with trypsin, it was purified to the MonoS fractions as described above, then concentrated and separated by SDS-PAGE. In order to be sure that the protein was chitinosanase, a proportion of the gel was submitted to chitinosanase activity staining. Next the chitinosanase bands were cut out directly from the gel and digested with trypsin. The peptides were extracted from the gel with acetonitrile and separated or sequenced by LC-MS. The following peptide sequences were identified:

| | |
|---|---|
| NLKVLLSIGGWSFSANFAGPASSDQK | (SEQ ID NO: 1) |
| DLNEDLLATPEK | (SEQ ID NO: 2) |

The present invention is not limited to embodiments described herein; reference should be had to the appended claims.

1-13. (canceled)

14. A chitinosanase obtainable from the fungus Alternaria alternate, the chitinosanase:
specifically cleaving a GlcNAc-GlcN glycosidic bond in a chitosan;
possessing a relative molecular weight as determined by SDS-PAGE of about 18 kDa;
having an optimum pH of about 4; and
having an optimum temperature of about 70° C.

15. The chitinosanase as recited in claim 14, wherein the chitinosanase is obtained from the Alternaria alternata strain CCT 2816 of the Coleção de Culturas Tropical, Brazil (DSM 22279).

16. The chitinosanase as recited in claim 14, wherein the chitinosanase has at least one of the protein fragments SEQ ID NO: 1 and SEQ ID NO: 2.

17. A DNA sequence that encodes a chitinosanase obtainable from the fungus Alternaria alternate, the chitinosanase
specifically cleaving a GlcNAc-GlcN glycosidic bond in a chitosan,
possessing a relative molecular weight as determined by SDS-PAGE of about 18 kDa,
having an optimum pH of about 4, and
having an optimum temperature of about 70° C.

18. A vector that contains a DNA sequence that encodes a chitinosanase obtainable from the fungus Alternaria alternate, the chitinosanase:
specifically cleaving a GlcNAc-GlcN glycosidic bond in chitosan,
possessing a relative molecular weight as determined by SDS-PAGE of about 18 kDa,
having an optimum pH of about 4, and
having an optimum temperature of about 70° C.

19. A host cell that has one or more of
a) a DNA sequence that encodes a chitinosanase obtainable from the fungus Alternaria alternate, the chitinosanase
specifically cleaving a GlcNAc-GlcN glycosidic bond in a chitosan,
possessing a relative molecular weight as determined by SDS-PAGE of about 18 kDa,
having an optimum pH of about 4, and
having an optimum temperature of about 70° C., and
b) a vector that contains the DNA sequence that encodes the chitinosanase so as to at least one of transform and transfect the host cell.

20. A method for producing a chitinosanase obtainable from the fungus Alternaria alternate, the chitinosanase
specifically cleaving a GlcNAc-GlcN glycosidic bond in a chitosan,
possessing a relative molecular weight as determined by SDS-PAGE of about 18 kDa,
having an optimum pH of about 4, and
having an optimum temperature of about 70° C., the method comprising:
cultivating host cells that have one or more of a) at least one of transformed and transfected with a vector that contains a DNA sequence that encodes the chitinosanase and b) the DNA sequence that encodes the chitinosanase; and
isolating the chitinosanase from at least one of the cultivated host cells and from a culture solution.

21. An enzyme or pharmaceutical composition comprising a chitinosanase obtainable from the fungus Alternaria alternate, the chitinosanase
specifically cleaving a GlcNAc-GlcN glycosidic bond in a chitosan,
possessing a relative molecular weight as determined by SDS-PAGE of about 18 kDa,
having an optimum pH of about 4, and
having an optimum temperature of about 70° C.

22. The enzyme or pharmaceutical composition as recited in claim 21, wherein the enzyme or pharmaceutical composition further comprises a (partially acetylated) chitosan.

23. A wound dressing containing the enzyme or pharmaceutical composition as recited in claim 22.

24. A method of degradation of a chitosan, the method comprising:
reacting the chitosan with a chitinosanase obtainable from the fungus Alternaria alternate, the chitinosanase
specifically cleaving a GlcNAc-GlcN glycosidic bond in a chitosan,
possessing a relative molecular weight as determined by SDS-PAGE of about 18 kDa,
having an optimum pH of about 4, and
having an optimum temperature of about 70° C.,
with at least one of 1) a host cell that has one or more of a) a DNA sequence that encodes the chitinosanase and b) a vector that contains the DNA sequence that encodes the chitinosanase so as to at least one of transform and transfect the host cell and 2) an enzyme or pharmaceutical composition comprising the chitinosanase, so as to obtain chitosan degradation products; and
isolating the chitosan degradation products.

25. Chitosan degradation products obtained by reacting the chitosan with a chitinosanase obtainable from the fungus Alternaria alternate, the chitinosanase
specifically cleaving a GlcNAc-GlcN glycosidic bond in a chitosan,
possessing a relative molecular weight as determined by SDS-PAGE of about 18 kDa,
having an optimum pH of about 4, and
having an optimum temperature of about 70° C.,
with at least one of 1) a host cell that has one or more of a) a DNA sequence that encodes the chitinosanase and b) a vector that contains the DNA sequence that encodes the chitinosanase so as to at least one of transform and transfect the host cell and 2) an enzyme or pharmaceutical composition comprising the chitinosanase, so as to obtain chitosan degradation products, and isolating the chitosan degradation products.

26. Method of using at least one of a) a chitinosanase obtainable from the fungus Alternaria alternate, the chitinosanase
specifically cleaving a GlcNAc-GlcN glycosidic bond in chitosan,
possessing a relative molecular weight as determined by SDS-PAGE of about 18 kDa,
having an optimum pH of about 4, and
having an optimum temperature of about 70° C., and b) an enzyme or
pharmaceutical composition comprising the chitinosanase, the method comprising:
providing at least one of the chitinosanase and the enzyme or pharmaceutical composition; and
incorporating the at least one of the chitinosanase and the enzyme or pharmaceutical composition in a wound dressing.

27. A method of treating a wound, the method comprising:
providing a wound dressing that comprises a) a chitinosanase obtainable from the fungus Alternaria alternate, the chitinosanase:
specifically cleaving a GlcNAc-GlcN glycosidic bond in a chitosan,
possessing a relative molecular weight as determined by SDS-PAGE of about 18 kDa,
having an optimum pH of about 4, and
having an optimum temperature of about 70° C., and b) a (partially acelylated) chitosan; and
applying the wound dressing on a wound of a patient so as to treat the patient.


SEQUENCE LISTING (2 sequences)

SEQ ID NO: 1 - protein, 26 aa, Alternaria alternata
NLKVLLSIGGWSFSANFAGPASSDQK

SEQ ID NO: 2 - protein, 12 aa, Alternaria alternata
DLNEDLLATPEK